(19) United States
(12) Patent Application Publication
(10) Pub. No.: US 2008/0031811 A1
(43) Pub. Date: Feb. 7, 2008

(54) Method For Preparing Radioactive Film

(75) Inventors: Jei-Man Ryu, Kyunggi-do (KR); Yu-Eun Kim, Kyunggi-do (KR); Seung-Kyoo Seong, Kyunggi-do (KR); Dong-Hyuk Shin, Kyunggi-do (KR); Byung-Ho Cho, Seoul (KR); Young-Jun Song, Seoul (KR); Kyung-Bae Park, Taejeon-si (KR); Byung-Chul Shin, Taejeon-si (KR)

Correspondence Address: LUCAS & MERCANTI, LLP, 475 PARK AVENUE SOUTH, 15TH FLOOR, NEW YORK, NY, 10016, US

(73) Assignees: DONG WHA PHARM. IND. CO., LTD., Seoul (KR); KOREA ATOMIC ENERGY RESEARCH INSTITUTE, Daejeon (KR)

(21) Appl. No.: 11/575,170

(22) PCT Filed: Sep. 12, 2005
(86) PCT No.: PCT/KR05/02999

§ 371 Date: Mar. 13, 2007

(30) Foreign Application Priority Data
Sep. 15, 2004 (KR) 10-2004-0073985

(51) Int. Cl. A61K 51/12; A61M 36/14

(52) U.S. Cl. 424/1.11; 252/634; 427/5

(57) ABSTRACT

The present invention relates to a method for preparing a radioactive film for local radioactive treatment. More particularly, the present invention relates to a method for preparing a radioactive film comprising the steps of; dissolving 0.1˜14.5 weight % of a stable nuclide and 13˜32.5 weight % of a film-forming base for the total amount of a solvent in the solvent; applying a stable nuclide solution on a release paper by a coater and drying; and irradiating a stable nuclide film with neutrons in a nuclear reactor. A method for preparing a radioactive film according to the present invention provides a radioactive film having a uniform distribution of radionuclides and an even thickness. Therefore, the therapeutic efficacy of the radioactive film for selective treatment of a lesion may be maximized by attaching the radioactive film on a patient's skin or a mucous membrane and by direct radioactive radiation.

### TECHNICAL FIELD

The present invention relates to a method for preparing a radioactive film for local radioactive treatment.

### BACKGROUND ART

The present invention relates to a method for preparing a radioactive film for local radioactive treatment. More particularly, the present invention relates to a method for preparing a radioactive film comprising the steps of; dissolving 0.1˜14.5 weight % of a stable nuclide and 13˜32.5 weight % of a film-forming base for the total amount of a solvent in the solvent; coating a stable nuclide solution on a release paper by a coater and drying; and irradiating a stable nuclide film with neutrons in a nuclear reactor. A method for preparing a radioactive film according to the present invention provides a radioactive film having a uniform distribution of radionuclides and a uniform thickness. Therefore, the therapeutic efficacy of a radioactive film for selective treatment of a lesion may be maximized by attaching the radioactive film on a patient's skin or a mucous membrane and by direct radioactive radiation.

During researches to find a way of selective application of radioactive materials to a lesion, the inventors have invented a radioactive patch/film and then the radioactive patch/film is directly attached on a patient's lesion for selective radioactive radiation(Korea Patent No. 170811).

However, the disclosed radioactive patch/film is prepared by applying stable nuclide particles on a supporter or coating a predetermined amount of a stable nuclide solution on a glass dish, then drying and separating the patch/film. Therefore, this method is not suitable for mass production. The treatment efficiency is low because the thickness of the prepared radioactive film and the distribution of radionuclides are not uniform. In addition, a residual solvent may cause skin irritation. An efficient method for the preparation of a radioactive film is required because these disadvantages are barriers for clinical applications.

The inventors have discovered a method to solve the above problems by changing a film base material and composition of a solvent and completed the present invention by preparing a radioactive film which has a uniform thickness, a uniform distribution of radionuclides and a very low amount of a residual solvent.

### DISCLOSURE OF THE INVENTION

### TECHNICAL PROBLEM

An object of the present invention is to provide a radioactive film that has a uniform thickness, a uniform distribution of radionuclides and a very low amount of a residual solvent.

Another object of the present invention is to provide a therapeutic agent for skin diseases including various skin cancers, dermatophytosis, and Keloid by using a radioactive film prepared by the above preparation method.

Another object of the present invention is to provide a composition of a stable nuclide solution for the preparation of a radioactive film.

### TECHNICAL SOLUTION

In order to solve the above technical problem, the present invention provides a method for preparing a radioactive film comprising the steps of; dissolving 0.1˜14.5 weight % of a stable nuclide and 13˜32.5 weight % of a film-forming base for the total amount of a solvent in the solvent; applying a stable nuclide solution on a release paper by a coater and drying; and irradiating a stable nuclide film with neutrons in a nuclear reactor.

In order to solve another technical problem, the present invention provides a therapeutic agent for skin diseases including various skin cancers, dermatophytosis, and Keloid by using a radioactive film prepared by the above preparation method.

In order to solve another technical problem, the present invention provides a composition of a stable nuclide solution for the preparation of a radioactive film that contains 0.1˜14. 5 weight % of a stable nuclide and 13˜32.5 weight % of a film-fbrming base for the total amount of a solvent.

### BRIEF DESCRIPTION OF DRAWINGS

FIGS. 1 to FIG. 7 are photographs showing surfaces of holmium-165 polyurethane films prepared in accordance with the example embodiments of the present invention, wherein

FIG. 1 is a photograph showing a film prepared by coating a preparatory solution of Example 3 on a dish;

FIG. 2 is a photograph showing a film prepared from a preparatory solution of Example 3 using a coater;

FIG. 3 is a photograph showing a film prepared from a preparatory solution of Example 4 using a coater;

FIG. 4 is a photograph showing a film prepared from a preparatory solution of Example 5 using a coater;

FIG. 5 is a photograph showing a film prepared from a preparatory solution of Example 22 using a coater;

FIG. 6 is a photograph showing a film prepared from a preparatory solution of Example 23 using a coater; and

FIG. 7 is a photograph showing a film prepared from a preparatory solution of Example 18 using a coater.

FIG. 8 shows a holmium-165 polyurethane film observed by an electron microscope.

FIG. 9 shows a graph of TGA result of a holmium-165 polyurethane film, wherein a) is a holmium-165 polyurethane film; b) is holmium nitrate; and c) is a polyurethane film.

FIG. 10 shows a graph of DSC result of a holmium-165 polyurethane film, wherein a) is a holmium-165 polyurethane film; b) is holmium nitrate; and c) is a polyurethane film.

FIG. 11 shows photographs of skin irritation results of a holmium-166 polyurethane film to hairless mice wherein 1) is a pathological appearance of a hairless mouse's normal skin tissue; 2) is a pathological appearance of a skin tissue damaged by radioactive radiation after attachment of the film (2-2: after 1 week of attachment, 2-3: after 2 weeks of attachment); 3) is a pathological appearance of the recovery and re-epithelization of a damaged skin tissue after 6 weeks of attachment of the film.

FIG. 12 shows photographs of treatment efficacy of a holmium-166 polyurethane film for hairless mice wherein 1) are pathological photos of a hairless mouse having an induced skin cancer and tumor tissues, and 2) shows the disappearance of the skin cancers after 6 weeks of the attachment.

FIGS. 13 and 14 show the result of clinical treatment of a holmium-166 polyurethane film for Kaposi's sarcoma patient, wherein FIG. 13 shows an appearance before the treatment and FIG. 14 shows an appearance after the treatment.

### BEST MODE FOR CARRYING OUT THE INVENTION

The object of the present invention is accomplished by providing a method for preparing a radioactive film comprising the steps of; dissolving 0.1˜14.5 weight % of a stable nuclide and 13˜32.5 weight % of a film-forming base for the total amount of a solvent in the solvent (step 1); applying a stable nuclide solution on a release paper by a coater and drying (step 2); and irradiating a stable nuclide film with neutrons in a nuclear reactor (step3).

In the step 1 of the method for preparing a radioactive film, 0.1˜14.5 weight % of a stable nuclide and 13˜32.5 weight % of a film-forming base for the total amount of a solvent are uniformly dissolved in the solvent.

Even in the case that radioactive materials cause a chemical change with film-forming bases, base materials used for the present invention do not have influence on exposure dose, and therefore most of conventional film-forming materials can be used without limitation.

Film-forming bases which can be used in the present invention are synthetic polymers such as polyethylene series, polypropylene series, polyester series, polyurethane series, polyvinyl chloride series, polyethylene tetrachloride series, polymethylmethacrylate series, polyglycolic acid series and nylon series; natural polymers such as collagen, chitosan, gelatin, and cellulose series and other base materials which can form a film.

A polyurethane elastomer is especially preferable due to the extensibility of rubber, flexibility and elasticity of film, resistance against mold and fungi, and resistance to radioactive rays. Additionally, a medical-grade polyurethane elastomer is more preferable when considering a biological safety.

13˜32.5 weight % of a film-forming base for the total amount of a solvent is added and 14˜25 weight% of a film-forming base is preferably added. When the amount of a base material is less than 13 weight %, the fluidity of a preparatory solution is high. Thus the preparation of a film having a uniform thickness is difficult due to the partial inclination of the solution before drying during the coating of a solution to get a predetermined thickness. Because thick application and drying of the preparatory solution is required to obtain a film that has the same thickness as the film prepared from highly concentrated solution, more organic solvent is required. Increase of the organic solvent amount causes problems such as skin irritation and low manufacturing efficiency due to the increased drying time. When the used amount of a base is above 32.5 weight %, the coating is irregular due to low fluidity of a solution. Evaporation of a solvent occurs on both the surface and inside of a solution. Thus the deviation of film thickness occurs because the distribution of the solution is not even due to the irregular distribution of concentration during coating. Time required to dissolve the base is 3 to 4 times long when compared with dissolving 13 weight % of a base. This causes a problem that the concentration of the solution is increased because of evaporation of solvent during dissolving process.

A radionuclide which may be used in the present invention is any nuclide emitting therapeutic radioactive radiation such as α-ray emitting nuclide, β-ray emitting nuclide or γ-ray emitting nuclide, and can be chosen according to the purpose of treatment.

Especially, lantanide radionuclide such as ¹⁶⁵Dy, ¹⁶⁶Ho, ¹⁵³Sm, or ¹⁶⁹Er has a relatively short half-life and is a β-ray emitting nuclide emitting a low energy γ-ray. Because each of stable lantanide nuclide has a large neutron absorption cross-section, each of them has an advantage that a stable lantanide nuclide is readily transformed to a radionuclide by irradiating with neutrons in a nuclear reactor.

The inventors have found that in the case of a radioactive film using a β-ray emitting nuclide of ¹⁶⁶Ho, maximum 8 mm of tissue is damaged by an excessive radiation dose, and soft-tissues or bones are not damaged at all. This result confirms that the use of a β-ray emitting nuclide is an effective medicine with less side-effects (Korea Patent No. 170811).

A compound of radionuclide soluble in a solvent may be used as a compound of a stable nuclide. A compound such as ¹⁶⁴Dy (NO₃)₃, ¹⁶⁴DyCl₃, ¹⁶⁵Ho (No₃)₃, or ¹⁶⁵Ho (No₃)₃.5H₂O is especially preferable.

¹⁶⁵Ho (No₃)₃, or ¹⁶⁵Ho(No₃)₃.5H₂O may be more preferably used.

A nuclear reaction of ¹⁶⁵Ho(No₃)₃.5H₂O as a target nucleus by neutron bombardment in a nuclear reactor for the production of a radioactive isotope produces a β-decay nuclide of ¹⁶⁶Ho(holmium-166). The half-life of holmium-166 is relatively short as 26.8 hours. The average range of penetration into soft tissues is 2.1 mm and the maximum penetration is 8.7 mm. Thus the holmium-166 provides a radioactive therapeutic agent effective for the local treatment of skin diseases.

A compound of stable nuclide according to the preparation method of the present invention is added in an amount of 0.1˜14.5 weight % for the total weight of a solvent depending on the required amount of a stable nuclide, considering radiation dose after radioactive labeling. When the content of the stable nuclide is less than 0.1 weight %, it does not have therapeutic effect. When the content of the stable nuclide exceeds 14.5 weight %, coagulation occurs in a solution mixed with a base. When this solution is used to prepare a film, coagulated solution is irregularly coated. Namely, when the content of a stable nuclide is in excess of 14.5 weight %, it is not easy to prepare a film because a film formation of the base is interfered.

The viscosity of a stable nuclide solution prepared in the present invention is preferably 400˜80,000 mPas. When the viscosity of the solution is below 400 mPas, it is impossible to prepare a film having a uniform thickness because of inclination of a solution during film preparation. When the viscosity of the solution is above 80,000 mPas, the fluidity of the solution is so low that a uniform film is not obtained. The most preferable viscosity of a stable nuclide solution is 1,000˜10,000 mPas.

Any organic solvent that can dissolve a stable nuclide and film-forming base may be used as a solvent for dissolving the film-forming base and stable nuclide. It is preferable to choose a solvent that has short drying time during film preparation and minimized irritation of a skin or mucous membrane by a residual solvent. Ethanol, methanol, acetone, dimethylformamide, tetrahydrofuran, or dimethylsulfoxide, etc. may be used as a solvent.

Dimethylformamide (DMF; bp 153° C.), tetrahydrofuran (THF; bp 66° C.) or a mixed solvent of dimethylformamide and tetrahydrofuran is preferably used in a preparation method of the present invention. Additionally, the solubility of film-forming base and stable nuclide in dimethylformamide or tetrahydrofuran is good and each of them may be used as a single solvent. However, a mixed solvent of dimethylformamide and tetrahydrofuran is more preferably used to dissolve a film-forming base easily and minimize drying time after coating and the amount of residual solvent in a film after drying. The volume ratio of dimethylformamide and tetrahydrofuran is more preferably 1:6˜1:11. When the volume ratio of dimethylformamide and tetrahydrofuran is less than 1:6, the amount of residual solvent is too large when considering that the amount of residual solvent in general skin patch is about 3 μg/cm². When the volume ratio is more than 1:11, the solubility of a base decreases.

When the solution containing the same concentration of a film-forming base is dissolved respectively using dimethylformamide, tetrahydrofuran or a mixed solvent of dimethylformamide and tetrahydrofuran, the dissolving time is decreasing in the order of dimethylformamide, the mixed solvent of dimethylformamide and tetrahydrofuran, and tetrahydrofuran. In the case of 25.0 weight % of polyurethane elastomer, when dissolved in dimethylformamide, the viscosity of a preparatory solution is 8,730 mPas; when dissolved in tetrahydrofuran, the viscosity of a preparatory solution is 8,013 mPas; and in the case of the mixed solvent of dimethylformamide and tetrahydrofuran, the viscosity of a preparatory solution is 7,734 mPas. There is no significant difference in viscosity.

Inner air bubbles in a stable nuclide solution prepared in the step 1 are removed and then the solution is thinly coated on a release paper by a coater. Film is formed after drying the coated solution. The release paper to be used for uniform coating of the stable nuclide solution is a silicone coated paper, PET film, PE film, or any of release papers that can be used in the preparation of plasters. A silicone coated paper or PET film is preferably used.

The thickness of a film is preferably 20˜200 μm. In the case of the thickness of less than 20 μm, attachment of the film on a lesion is inconvenient and there is a limitation for containing sufficient amount of stable nuclide particles. In the case of the thickness of more than 200 μm, radioactive radiation from radionuclides located far away from a lesion do not reach a lesion, and thus a problem of unnecessary radioactive radiation occurs.

The dried stable nuclide film of the present invention is cut in a circle shape of 1 cm diameter or other required shape and size. A radioactive film for skin patch is prepared from the nuclear reaction by neutron bombardment in a nuclear reactor for the production of radioactive isotopes (for example, thermal neutron flux: 1.0×10¹³˜1.0×10¹⁴ n/cm²·sec, reaction time: 50 hrs., target material: holmium nitrate, nuclear reaction cross section: 64 barns, target container: aluminum can)

A radioactive film prepared in the present invention is used to skin diseases occurring on a skin or mucous membrane. It is very effective for the treatment of skin cancers such as basal cell carcinoma, squamous cell carcinoma, melanoma, kaposi's sarcoma, paget's disease, and mycosis fungoides.

Hereinafter, preferred example embodiments and experimental examples of the present invention will be described more fully. This invention may, however, be embodied in many different forms and should not be construed as limited to the example embodiments set forth herein.

### EXAMPLES 1˜24

### Preparation of Holmium-166(165) Polyurethane Film

1) Preparation of Holmium 165-Polyurethane Film

Preparatory solutions of holmium 165-polyurethane film are prepared separately according to the composition of Table 1.

Polyurethane elastomer is completely dissolved in a mixture solution of dimethylformamide (DMF) and tetrahydrofuran (THF), and then holmium nitrate (¹⁶⁵Ho(No₃)₃.5H₂O) is added to the solution. After holmium nitrate is completely dissolved, the mixture is left at room temperature until bubbles of the mixed solution disappear. A holmium 165-polyurethane film is prepared by coating a holmium 165-polyurethane solution prepared in accordance with each example embodiment on a release paper by a coater (Laboratory Drawdown coater LC-100, Chem Instruments Co.) with a predetermined thickness (100˜1500 μm), drying for few seconds by a heat-gun and then completely dried at room temperature. The dried film has a thickness of 20-200 μm.

2) Preparation of Holmium 166-Polyurethane Film

A holmium 165-polyurethane film is cut in a circle shape of 1 cm diameter. A radioactive film of holmium 166-polyurethane having 6˜8 mCi is prepared by irradiation of the above circle shape film as a target material in a nuclear reactor where the neutron flux is 4×10¹³(n/cm²·sec).

TABLE 1
Compositions of Examples 1˜24

| | | Weight % of | | | | |
|---|---|---|---|---|---|---|
| | Weight % of | holmium | | | | |
| | polyurethane | nitrate for | Composition | | | |
| | for the total | the total | | Holmium | | |
| | weight of a | weight of a | Polyurethane | nitrate | DMF¹⁾ | |
| Example | solvent | solvent | elastomer (g) | (g) | (mL) | THF²⁾ (mL) |
| 1 | 5.5 | 5.5 | 2.43 | 2.43 | 4.5 | 45 |
| 2 | 8 | 10 | 3.54 | 4.42 | 4.5 | 45 |
| 3 | 11.13 | 11.13 | 2.70 | 2.70 | 4.5 | 22.5 |
| 4 | 12.5 | 10 | 5.53 | 4.42 | 4.5 | 45 |
| 5 | 13 | 10 | 5.75 | 4.42 | 4.5 | 45 |
| 6 | 13.5 | 10 | 3.81 | 2.82 | 4.5 | 27 |
| 7 | 14 | 9 | 4.51 | 2.90 | 4.5 | 31.5 |
| 8 | 15 | 15 | 6.64 | 6.64 | 4.5 | 45 |
| 9 | 16 | 12 | 5.80 | 4.35 | 4.5 | 36 |
| 10 | 17 | 5 | 6.84 | 2.01 | 4.5 | 40.5 |
| 11 | 17 | 17 | 7.52 | 7.52 | 4.5 | 45 |
| 12 | 19 | 10 | 8.41 | 4.42 | 4.5 | 45 |
| 13 | 20 | 10 | 8.85 | 4.42 | 4.5 | 45 |
| 14 | 20 | 14.5 | 8.85 | 6.41 | 4.5 | 45 |
| 15 | 20 | 15 | 8.85 | 6.63 | 4.5 | 45 |
| 16 | 22 | 5 | 11.50 | 2.61 | 4.5 | 54 |
| 17 | 22 | 17 | 9.73 | 7.52 | 4.5 | 45 |
| 18 | 22 | 23 | 9.73 | 10.18 | 4.5 | 45 |
| 19 | 25 | 10 | 12.06 | 4.82 | 4.5 | 49.5 |
| 20 | 25.5 | 10 | 11.28 | 4.42 | 4.5 | 45 |
| 21 | 29 | 10 | 12.83 | 4.42 | 4.5 | 45 |
| 22 | 32.5 | 8 | 14.38 | 3.54 | 4.5 | 45 |
| 23 | 33 | 12 | 14.60 | 5.31 | 4.5 | 45 |
| 24 | 35 | 5 | 15.49 | 2.21 | 4.5 | 45 |

¹⁾d = 0.9445,
²⁾d = 0.8892

### EXPERIMENTAL EXAMPLE 1

### Determination of the Concentration of Polyurethane Elastomer

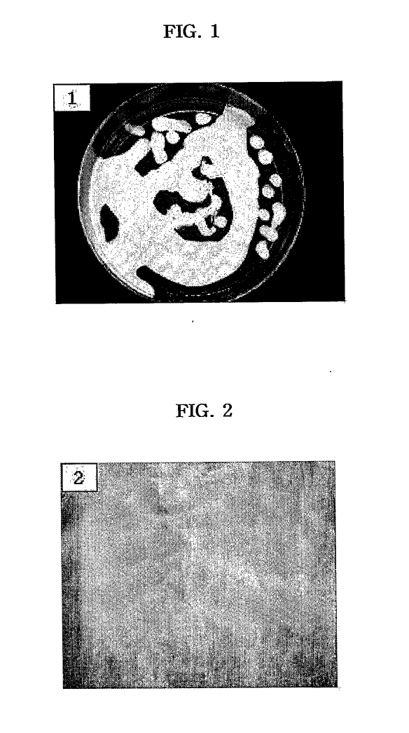

The optimum concentration of polyurethane elastomer is determined by comparing dissolving time of polyurethane elastomer, uniformity of a film, and drying time in the preparation of film having the composition of Examples 1˜24. The images of each film are taken by a digital camera (Casio EX-Z40) and shown in FIGS. 1 to 7.

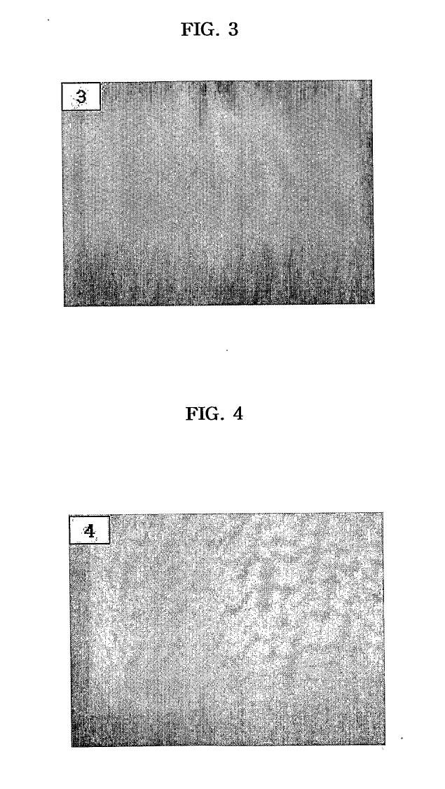

Films are prepared in accordance with Examples. The film with a uniform thickness is not obtained with a preparatory solution containing a polyurethane elastomer of less than 13 weight % (Examples 1˜4) because the fluidity of preparatory solution is high due to low concentration and inclination of solution occurs partially before drying when coating to a predetermined thickness (FIGS. 2 and 3). In order to obtain a dried film with the same thickness as a film prepared from high concentration solution, thick coating of the preparatory solution, long drying time, and use of a larger amount of solvent are required.

In the cases of Examples 5 and 20 where concentration of polyurethane elastomer is 13% and 25.5% respectively, the coated amount of Example 5 is two times more than Example 20 to obtain a film with the same dried thickness as Example 20. Thus the required time to remove the solvent from the film becomes long.

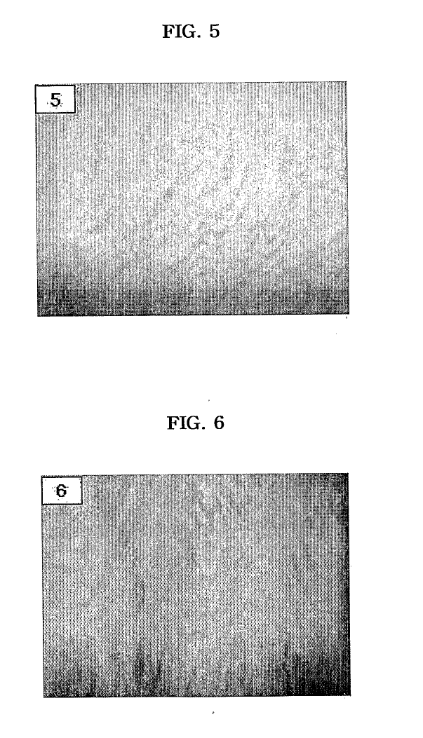

Film with a uniform thickness is prepared in a variety of thickness without an inclining phenomenon of solution during coating (FIGS. 4 and 5) when the concentration of polyurethane elastomer is more than 13 weight % (Examples 5˜22). However, when the concentration of polyurethane elastomer is in excess of 32.5 weight % (Examples 23 and 24), the fluidity of the preparatory solution is so low that the coating is irregular, and there is a difference in the thickness of films (FIG. 6) because the distribution of solution is not uniform during coating due to the evaporation of solvent in the inside and surface of the solution. Additionally, the dissolving time of a base is required 3 to 4 times longer than the dissolving time of 13weight %.

Therefore, the optimum concentration of polyurethane elastomer for the preparation of a film by using a film manufacturing machine without difficulty in dissolving of the preparatory solution is 13˜32.5 weight %, preferably 14˜25 weight %.

### EXPERIMENTAL EXAMPLE 2

### Determination of Concentration of Stable Nuclide

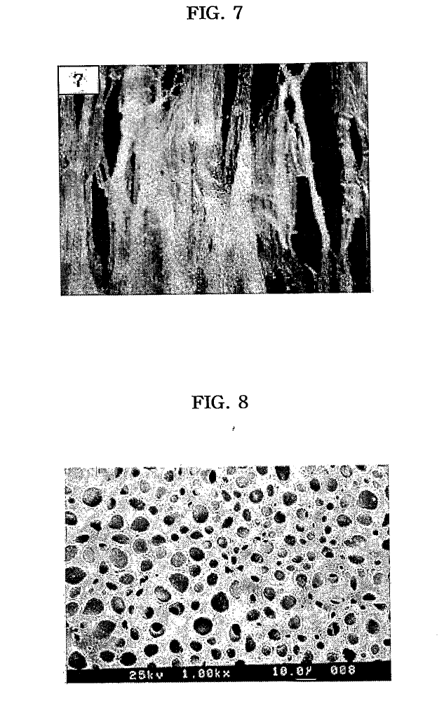

The optimum concentration of a stable nuclide compound is determined by observing uniformity of a film according to the concentration of a stable nuclide in the film prepared in Examples. The amount of a stable nuclide compound is 0.1˜14.5 weight % for the total weight of a solvent according to the required stable nuclide, considering radiation dose after radioactive labeling. In the cases that the amount is in excess of 14.5 weight % (Examples 8, 11, 15, 17, and 18), coagulation occurs in a solution when dissolving with a polyurethane elastomer, and the coagulated solution is coated unevenly when this solution is used to prepare a film (FIG. 7). Namely, when the content of the stable nuclide is in excess of 14.5 weight %, it is difficult to be used for the preparation of a film because the phenomenon interrupting the film formation of a base occurs.

### EXPERIMENTAL EXAMPLE 3

### Measurement of Viscosity of the Preparatory Solutions

The viscosity of a preparatory solution is measured by a viscometer (DV-II+ Viscometer, Brookfield Eng. Labs Inc.) and the results are summarized in Table 2. The preparation of a solution and film is easy in the range of viscosity of Examples 5 to 22 (except Example 15 in which only preparation of the solution is easy). Namely, the viscosity for the preparation of a solution and film is preferably 400˜80,000 mPas, and more preferably 1,000˜10,000 mPas. There are no significant changes in viscosity of the preparatory solution according to the addition of holmium nitrate as carried out in Examples 13, 14 and 15.

TABLE 2

| | Weight % of | Weight % of holmium | |
|---|---|---|---|
| | polyurethane for the | nitrate for the | |
| | total weight of | total weight of | Viscosity |
| Example | solvent | solvent | (mPas) |
| 2 | 8 | 10 | 77 |
| 3 | 11.13 | 11.13 | 219 |
| 4 | 12.5 | 10 | 325 |
| 5 | 13 | 10 | 404 |
| 6 | 13.5 | 10 | 707 |
| 7 | 14 | 9 | 1,012 |
| 13 | 20 | 10 | 4,719 |
| 14 | 20 | 14.5 | 4,693 |
| 15 | 20 | 15 | 4,702 |
| 19 | 25 | 10 | 7,734 |
| 20 | 25.5 | 10 | 12,447 |
| 21 | 29 | 10 | 45,503 |
| 22 | 32.5 | 8 | 78,484 |
| 23 | 33 | 12 | 85,200 |
| 24 | 35 | 5 | 104,069 |

### EXPERIMENTAL EXAMPLE 4

### Observation of Film Surface by a Scanning Electron Microscope

The surface of a holmium-165 polyurethane film (Example 13) is observed by a scanning electron microscope (SEM, Model ISI-SX-30E, Japan). The surface of the holmium-165 polyurethane film is gold plated by an ion coater (Ion Coater, EIKO IB 3) to prevent the accumulation of charges that is resulted from the collision between electron beam and a specimen. The photograph shows that regular pores on the surface of the film are distributed (FIG. 8).

### EXPERIMENTAL EXAMPLE 5

### Measurement of Thermal Characteristic of Film

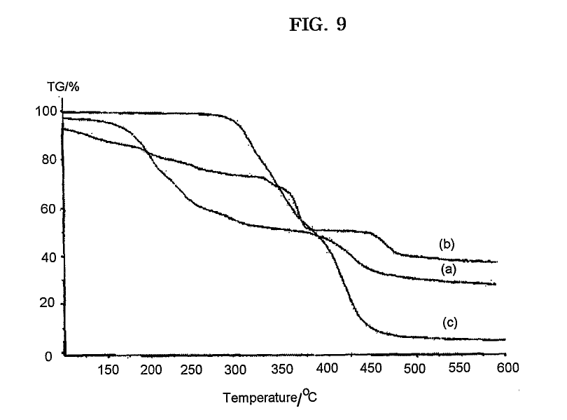

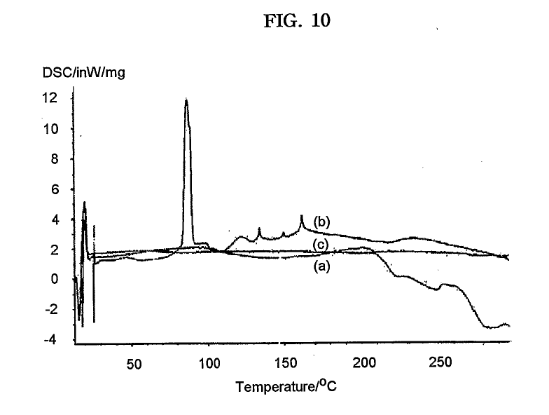

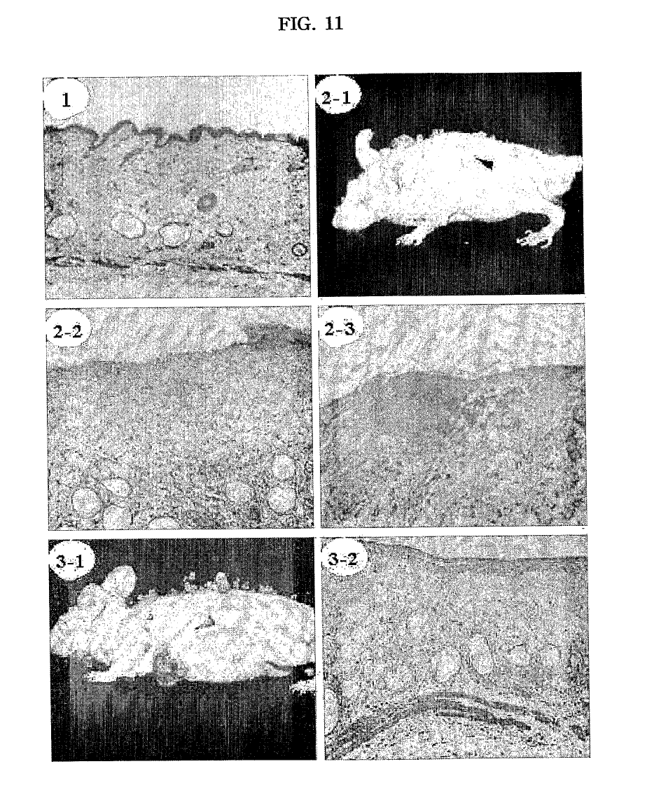

The behavior of thermal change according to temperature change in holmium nitrate, polyurethane film, and holmium-165 polyurethane film (Example 13) is measured by using a thermogravimetric analyzer (TGA, Netzsch DSC-204, Germany) and a differential scanning calorimetry (DSC, Netzsch TG-209, Germany). TGA analyzes the changes in the mass of a substance according to temperature by a thermal decomposition curve. The analysis conditions of TGA are as follows; 30˜600° C. of temperature, 5.0 mg of a substance, 10° C./min of heating rate. Calorie change of a substance according to temperature change is measured by DSC. DSC is measured at the same condition as TGA and the sample is sealed in an aluminum pan under nitrogen atmosphere. The results are shown in FIGS. 9 and 10.

According to the result measured by TGA, the decomposition of a polyurethane film begins at 297.3° C. and continues to 439.1° C. In the decomposition of holmium nitrate, nitrate group and water contained in the molecules decompose from 80° C. to 310° C., and two large decomposition curves are observed in the ranges of 345˜380° C. and 430˜480° C. The decomposition of holmium nitrate continues to 480° C.

In the case of holmium-165 polyurethane film, there is a gentle weight loss due to moisture and a solvent from 30° C. to 160° C. Decomposition curve due to the influence of holmium is shown from 176.9 to 290° C. Decomposition curves showing characteristics of polyurethane and holmium are observed in the ranges of 300˜340° C. and 340˜453° C. After decomposition, the residual weight % is measured at 500° C. Residual weight % of polyurethane film and holmium nitrate is 5% and 37% respectively. The residual weight % of holmium-165 polyurethane is 29% that is in the middle of that of the polyurethane film and holmium nitrate (FIG. 9).

The results of DSC measurements show that the curve for the polyurethane film is a very gentle slope without change according to the increase of temperature. In the case of holmium nitrate, there is an endothermic peak between 80˜100° C. due to a solvent. There is almost no peak change due to a calorie change in holmium-165 polyurethane film (FIG. 10). The behaviors of thermal change according to the temperature change show the intrinsic peaks of each substance in only TGA, and it is confirmed that polyurethane and holmium nitrate is physically mixed to form a holmium-165 polyurethane film.

### EXPERIMENTAL EXAMPLE 6

### Measurement of Mechanical Properties of Films

Mechanical properties of a polyurethane film and holmium-165 polyurethane film are measured by Material Testing System (Instron Co. Series IX).

The stress values of the polyurethane film and holmium-165 polyurethane film are 483.3±186.4 and 314.1±69.9 (kgf/cm²) respectively. The strain values of the polyurethane film and holmium-165 polyurethane film are 746.7±133 and 649±66.8% respectively. The stress value is the applied force per unit area of the film till the film is elongated and cut, and the strain value represents elongation. The stress and strain values of the holmium-165 polyurethane film are lower than those of the polyurethane film. This result indicates that the holmium-165 polyurethane film having a reduced elongation and strength is formed by adding holmium nitrate to polyurethane. The holmium nitrate capable of forming physical bonding with polyurethane is disposed between polyurethane molecules and forms a loose structure having a wide distance between molecules by adding holmium nitrate having lone pair of electrons between polyurethane molecules having regularly arranged structure. Therefore, a holmium-165 polyurethane film is softer than a polyurethane film

### EXPERIMENTAL EXAMPLE 7

### Measurement of Distribution of Holmium in Film

Holmium-165 polyurethane films (Examples 3, 4, 5, 7, 12, 19, 22, and 23) are cut from several places in a predetermined size (2.5×2.5 cm). Each piece is dissolved in the mixture solution of DMF and THF (1:10, v/v) respectively, filtered and the amount of holmium nitrate in the film is measured by a UV-Vis. Spectrophotometer (Hitech U 3000). The result is shown in Table 3.

The amount of holmium nitrate in a film according to Examples 3 and 4 is 1.028±0.524(mg/cm²) and 1.116±0.251(mg/cm²) respectively (standard deviation is more than 22%) and the distribution of holmium differs according to the location of the film. The amount of holmium nitrate in a film according to Example 23 is 1.969±1.269 (mg/cm²) (standard deviation is more than 60%) and the distribution of holmium content in the film is not uniform due to difference in thickness of uneven film. On the other hand, in the film of Examples 5, 7, 12, 19 and 22 in which production of uniform film is possible (Experimental Example 1), the standard deviations are below 9%. This result shows that holmium nitrate is relatively evenly distributed in a whole film. Therefore, It is judged that this film shows steady efficacy because radioactivity is evenly distributed in a holmium-166 polyurethane film after radioactive radiation, and β-ray as a therapeutic radiation is evenly transmitted when the film is attached on a lesion.

TABLE 3

| | | | Content of |
|---|---|---|---|
| | Weight % of | Weight % of | holmium nitrate |
| | polyurethane for | holmium nitrate | Avg. ± |
| | the total weight | for the total | S.D (mg/cm²), |
| Example | of solvent | weight of solvent | N = 5 |
| Example 3 | 11.13 | 11.3 | 1.028 ± 0.524 |
| Example 4 | 12.5 | 10 | 1.116 ± 0.251 |
| Example 5 | 13 | 10 | 1.353 ± 0.116 |
| Example 7 | 14 | 9 | 1.461 ± 0.065 |
| Example 12 | 19 | 10 | 1.903 ± 0.009 |
| Example 19 | 25 | 10 | 1.643 ± 0.094 |
| Example 22 | 32.5 | 8 | 1.325 ± 0.112 |
| Example 23 | 33 | 12 | 1.969 ± 1.269 |

### EXPERIMENTAL EXAMPLE 8

### Measurement of Amount of a Residual Solvent in Film

Holmium-165 polyurethane films (Examples 3, 6, 7, 9, 10, 12, 16, and 19) are cut in a predetermined size. After the cut film is completely dissolved in THF or DMF, the residual amount of DMF and THF in the film is analyzed by GC(Hewlett Packard 5890, Series II), respectively. The results are summarized in Table 4. The residual amount of DMF in the film of Example 3 (DMF:THF=1:5) is 2.43˜3.73 (μg/cm²). The residual amount of DMF in the films of Examples 6, 7, 9, 10, 12, 16, and 19 (DMF:THF=1:6˜12) is 0.68˜3.07 (μg/cm²). In the case of THF, the residual amount in the film is almost constant in the range of 0.15˜0.35 (μg/cm²) regardless of the increase in the use ratio.

When the ratio of DMF and THF in the mixed solvent is varied from 1:5 to 1:12, the amount of a residual organic solvent, especially DMF is decreased after drying and harmful effect of the organic solvent is reduced when the film is attached on a normal skin or a lesion. Because a very small amount of solvent is remained in a holmium-165 film prepared in the mixed solvent in which the ratio of DMF and THF is more than 1:6, it is concluded that there is no detrimental effect when considering that the amount of residual solvent in general skin patch dosage form is 3 μg/cm². When the ratio of DMF and THF in the mixed solvent is 1:12, the amount of residual solvent is low. However, the solubility of a base in the above mixed solvent is decreased. Therefore, the optimum volume ratio of DMF and THF is 1:6˜11, preferably 1:9˜11 to easily prepare the film and minimize the amount of a residual solvent.

TABLE 4

| | |
|---|---|
| | Amount of a |
| | residual solvent |
| DMF:THF volume | (μg/cm²) |

| Example | ratio | DMF | THF |
|---|---|---|---|
| 3 | 1:5 | 2.43˜3.73 | 0.17˜0.28 |
| 6 | 1:6 | 1.89˜3.07 | 0.19˜0.27 |
| 7 | 1:7 | 1.53˜2.87 | 0.19˜0.25 |
| 9 | 1:8 | 1.09˜2.85 | 0.15˜0.29 |
| 10 | 1:9 | 0.70˜2.45 | 0.16˜0.31 |
| 12 | 1:10 | 0.69˜2.31 | 0.19˜0.30 |
| 19 | 1:11 | 0.68˜2.12 | 0.21˜0.33 |
| 16 | 1:12 | 0.69˜1.95 | 0.22˜0.35 |

### EXPERIMENTAL EXAMPLE 9

### Evaluation of a Skin Irritation of Holmium-166 Polyurethane Film

An imaginary line is drawn from the head to tail of a hairless mouse for the evaluation of skin irritation of a holmium-166 polyurethane film (Example 13) for a hairless mouse. Efficacy evaluation is carried out by inducing skin cancers on the right back of the mouse, and a skin irritation experiment is carried out on the normal left back of the mouse.

A holmium-166 polyurethane film (0.6 mCi) having a circle shape of 5 mm diameter is applied on the left back for 2 hours and then skin irritation is evaluated after removal of the film. Any abnormal manifestation is not observed in total 13 Examples after 1 day passed. Abnormal manifestation is not observed after 3 days passed except that light erythema is observed in 3 cases. After 1 and 2 weeks of the application of the holmium-166 polyurethane film, skin redness is deepened by radioactive radiation, and erosion and ulcer occur (2 in FIG. 11). However, they become cured as time passes, and are completely disappeared after 6 weeks. Histopathologically damaged region is restored by re-epithelization (3 in FIG. 11). Namely, when enough amount of the holmium-166 polyurethane film is applied for the treatment of skin cancers, the ulcer and dermatitis induced by radioactive radiation are completely vanished and the skin is restored by re-epithelization as time passes.

### EXPERIMENTAL EXAMPLE 10

### Measurement of Therapeutic Efficacy of Holmium-166 Polyurethane Film

1) Induction of Skin Cancers of Animal Models

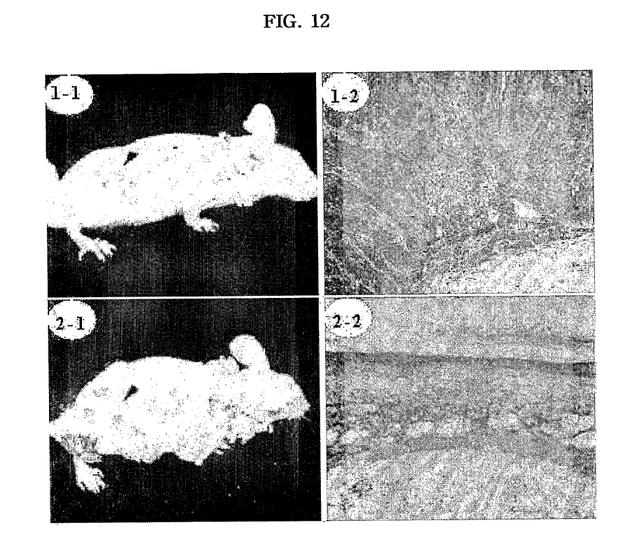

An imaginary line is drawn on the back of a hairless mouse from the head to tail. Skin cancers are induced by treating DMBA(7,12-dimethylbenz-α-anthracene) solution and TPA(12-tetradecanoyl-phorbol-13-acetate) solution in turn one time per a day for 15 weeks. DMBA is used as an initiator and dissolved in dimethylsulfoxide in the concentration of 1 mg/ml. TPA acts as a promoter and is dissolved in acetone to be 2 μM. After 8 weeks of the application of DMBA and TPA, a benign tumor of keratoacanthoma appears. A malignant carcinoma is observed in a plurality of cases at 12 weeks after the treatment. The tumors grow larger between 15 to 16 weeks and the infiltration of tumor cells to hypodermic tissues is observed. In contrast to the benign tumor of keratoacanthoma, squamous cell carcinoma is increased in an irregular form, and the infiltration of tumor cells to the derma and keratin pearl are characteristically observed (1 in FIG. 12).

2) The Treatment of Skin Cancers

Skin cancers are induced on the right back of hairless mice. The skin cancers are treated for 2 hours with a holmium-166 polyurethane film (0.6 mCi) having a circle shape of 5 mm diameter, with the composition of Example 13. The histopathological changes of tumors by radioactive radiation are observed after the films are applied to the skin cancers and the mice are sacrificed at 1, 2, 3, and 6 weeks.

The disappearance of tumors is observed at 1 and 2 weeks after the treatment and necrosis of tumors is observed after 3 weeks. After 6 weeks the growth of tumors is impossible because tumors are removed. It is identified that all of the tumor cells are histopathologically vanished (2 in FIG. 12).

### EXPERIMENTAL EXAMPLE 11

### Measurement of Clinical Therapeutic Efficacy of Holmium-166 Polyurethane Film

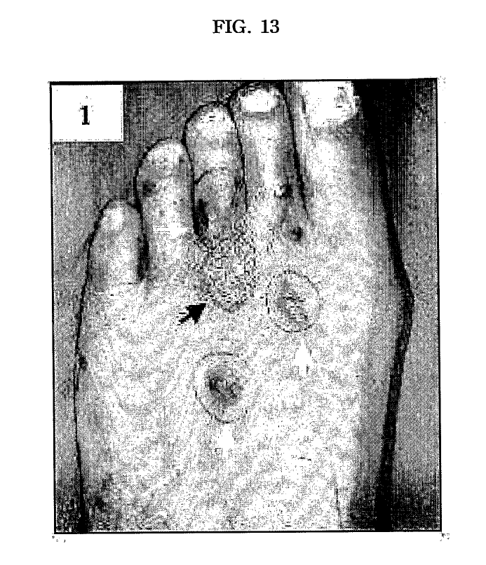

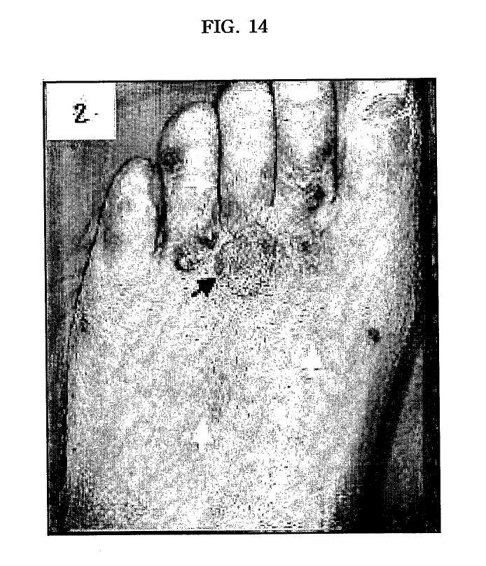

The four lesions in Kaposi sarcoma patient are treated by applying a holmium-166 polyurethane film and by conventional surgical resection (FIG. 7, 1; before treatment, 2; after treatment). The cured areas ( [symbol] in FIGS. 13 and 14) treated by the application of the holmium-166 polyurethane film (Example 13) are perfectly healed without any damage of tissues shown in the surgical resection ( [symbol] in FIGS. 13 and 14). The result of the treatment shows an excellent therapeutic efficacy with the short regeneration time of tissues and an advantage in beauty during treatment process.

### INDUSTRIAL APPLICABILITY

A radioactive film according to the present invention is prepared by radioactive radiation of a film containing a stable nuclide. This method may reduce exposure level of workers, and provide a simple manufacturing process and a high manufacturing efficiency suitable for mass production. Because the thickness of the prepared radioactive film and the distribution of radionuclides are uniform, the selectivity for lesions and treatment efficacy are improved greatly, and almost no skin irritation is shown due to reduced residual solvent. This convenient, stable and effective skin patch radioactive film may provide a useful healing method for the treatment of skin diseases.

1. A preparation method for a radioactive film comprising the steps of;
(1) dissolving 0.1˜14.5 weight % of a stable nuclide and 13˜32.5 weight % of a film-forming base for the total amount of a solvent in the solvent;
(2) applying a stable nuclide solution prepared in the step (1) on a release paper by a coater and drying;
(3) irradiating a stable nuclide film prepared in the step(2) with neutrons in a nuclear reactor.

2. The preparation met-hod of a radioactive film according to claim 1, wherein the stable nuclide is selected from the group consisting of ¹⁶⁴Dy(NO₃)₃, ¹⁶⁴DyCl₃, ¹⁶⁵Ho(No₃)₃, and ¹⁶⁵[Ho(No₃)₃.5H₂O].

3. The preparation method of a radioactive film according to claim 2, wherein the stable nuclide is selected from the group consisting of ¹⁶⁵Ho(No₃)₃, and ¹⁶⁵[Ho(No₃)₃.5H₂O]

4. The preparation method of a radioactive film according to claim 1, wherein the film-forming base is a medical-grade polyurethane elastomer.

5. The preparation method of a radioactive film according to claim 1, wherein the solvent is selected from the group consisting of dimethylformamide, tetrahydrofuran, and a mixture solution of dimethylformamide and tetrahydrofuran.

6. The preparation method of a radioactive film according to claim 5, wherein the solvent is a mixture solution of dimethylformamide and tetrahydrofuran in the range of volume ratios from 1:6 to 1:11.

7. The preparation method of a radioactive film according to claim 1, wherein the viscosity of the stable nuclide solution prepared in the step (1) is 400˜80,000 mPas.

8. The preparation method of a radioactive film according to claim 1, wherein the thickness of the radioactive film is 20˜200 μm.

9. The preparation method of a radioactive film according to claim 1, wherein the radioactive film is cut in a circle shape having various diameters or corresponding to a treated region.

10. A therapeutic agent for skin diseases including various skin cancers, dermatophytosis and Keloid by using the radioactive film prepared according to the claim 1.

11. A composition of a stable nuclide solution for the preparation of a radioactive film including 0.1˜14.5 weight % of a stable nuclide and 13˜32.5 weight % of a film-forming base for the total amount of a solvent dissolved in the solvent.

12. The composition of a stable nuclide solution for the preparation of a radioactive film according to claim 11, wherein the solvent is selected from the group consisting of dimethylformamide, tetrahydrofuran, and a mixture solution of dimethylformamide and tetrahydrofuran.

13. The composition of a stable nuclide solution for the preparation of a radioactive film according to claim 12, wherein the solvent is a mixture solution of dimethylformamide and tetrahydrofuran in the range of volume ratios from 1:6 to 1:11.

14. The composition of a stable nuclide solution for the preparation of a radioactive film according to claim 11, wherein the viscosity of the stable nuclide solution is 400˜80,000 mPas.
